(19) United States
(12) Patent Application Publication
(10) Pub. No.: US 2026/0365959 A1
(43) Pub. Date: Nov. 19, 2026

(54) DUAL FUNCTIONAL PIEZOELECTRIC DEVICE FOR INCREASING THE EFFICIENCY OF FERMENTATION PROCESSES

(71) Applicant: Danmarks Tekniske Universitet, Kongens Lyngby (DK)

(72) Inventors: Bhaskar Reddy SUDIREDDY, Kongens Lyngby (DK); Simone SANTUCCI, Kongens Lyngby (DK)

(73) Assignee: Danmarks Tekniske Universitet, Kongens Lyngby (DK)

(21) Appl. No.: 19/487,798

(22) PCT Filed: May 31, 2024
(86) PCT No.: PCT/EP2024/064976

§ 371 Date: Nov. 25, 2025

(30) Foreign Application Priority Data
Jun. 1, 2023 (EP) 23176686.6

(51) Int. Cl. C12M 1/42; C12M 1/00; C12M 1/12; C12N 1/20; C12N 13/00; H10N 30/00; H10N 30/50

(52) U.S. Cl. CPC C12M 35/02; C12M 23/06; C12M 23/20; C12M 23/58; C12M 35/04; C12M 37/00; C12N 1/20; C12N 13/00; H10N 30/50; H10N 30/702

(57) ABSTRACT

A fermentation enhancement device for use in a bioreactor, the fermentation enhancement device including: an electrical charge structure defining an inner volume arranged for accommodating a fermentation process, the electrical charge structure including: a piezoelectric proliferation layer exposed to the inner volume; and a piezoelectric virucidal layer arranged at an outer surface of the electrical charge structure; an opening configured to allow therethrough a flow of nutrients and/or bacteria. The fermentation enhancement device is arranged such that actuation of the electrical charge structure, by an actuation source, generates an electrical charge at the virucidal layer that kills and/or inactivates viruses, and an electrical charge at the proliferation layer that stimulates bacterial growth.

### CROSS-REFERENCE TO RELATED APPLICATIONS

The present application is the US national phase entry of International Patent Application No. PCT/EP2024/064976, filed May 31, 2024, which claims priority to European Patent Application No. 23176686.6, filed Jun. 1, 2023, the entire contents of both are hereby incorporated by reference.

### TECHNICAL FIELD

The present disclosure relates to dual functional piezoelectric devices and its use in fermentation processes.

### BACKGROUND

Fermentation accounts for a substantial proportion of human foods, including not only fermented fish, meat, vegetables, legumes, staple foods such as bread, cereal porridges and fermented coffee, cocoa, and condiments such as vinegar, soy sauce, and fish sauces but also alcoholic beverages and dairy.

The manufacture of fermented products requires the inoculation of selected bacterial cells, for example, dairy products require lactic acid bacteria (LAB). LAB digests glucose to produce lactic acid, turning milk into yogurt, cheese, or butter. The amount of lactic acid produced per unit of glucose is called yield and describes how efficient the acidification of the milk is.

Bacteriophages (also known as phages) are a family of viruses specialized in killing bacteria, including LAB. They are fundamentally ubiquitous and cause never-ending problems in the fermentation process, particularly in the dairy industry. During fermentation, phages can infect/disable LAB, and drastically decrease (or stop) the acidification process, resulting in low-quality or wasted product. Several solutions have been proposed to avoid phage action, but they substantially increase the production expenses (e.g. ultra sterile equipment, phage-resistant LAB strains), can lower the quality of the end-product (LAB rotation), and do not guarantee complete protection (even in a sterile environment, the wasted production is at least 10%).

Electrical charges interact with bacteria such as LAB and are shown to increase the redox process of some bacteria, resulting in a higher growth or higher product yield. On the other hand, a high population of charges triggers reactive oxygen species (ROS) formation. ROS interact with bacteria and are reported to disrupt their membranes, thereby killing them. Electrical cues have been studied both to increase bacteria growth (electro-fermentation) and for bactericidal effects.

### SUMMARY

A purpose of the present disclosure is to provide a simpler, affordable, and reliable solution to improve the efficiency of fermentation processes by both increasing bacteria production yield and inactivating and/or destroying potentially contaminating bacteriophages.

The present disclosure, therefore, relates to a fermentation enhancement device for use in a bioreactor, the fermentation enhancement device comprising:
an electrical charge structure defining an inner volume arranged for accommodating a fermentation process, the electrical charge structure comprising:
   i. a piezoelectric proliferation layer exposed to the inner volume; and
   ii. a piezoelectric virucidal layer arranged at an outer surface of the electrical charge structure;
an opening configured to allow therethrough a flow of nutrients and/or fermenting microorganism;
   wherein the fermentation enhancement device is arranged such that actuation of the electrical charge structure, by an actuation source, generates an electrical charge at the virucidal layer that kills and/or inactivates viruses and/or microorganisms, and an electrical charge at the proliferation layer that stimulates growth of fermenting microorganisms.

The presently disclosed fermentation enhancement device is arranged to be used for fermentation of a bacteria culture, such as LAB. The presently disclosed fermentation enhancement device is also suitable for fermentation of eukaryotic microorganisms, such as fungi (for example Saccharomyces cerevisiae and other S. species or other genera; Penicillium sp., Aspergillus sp., Rhizopus sp. and/or Geotrichum sp., as well as other yeasts and/or filamentous fungi), and/or algae such as Chlorella sp., Scenedesmus sp., Desmodesmus sp., Microcystis sp., as well as other algae and microalgae. The fermentation enhancement device is typically used in combination with an actuation source, for actuation of the layers of the electrical charge structure, and a fermentation vessel arranged to accommodate the fermentation enhancement device and a fermentation solution comprising the medium and fermenting microorganism.

During use, the fermentation enhancement device is arranged to generate electrical charges both at the proliferation layer and at the virucidal layer, and in particular electrical charges at the proliferation layer that increase growth of a fermenting microorganism and electrical charges at the virucidal layer that have a virucidal and/or fungicidal and/or bactericidal effect. In this way, the presently disclosed fermentation enhancement device relies on the ability of piezoelectric materials to generate specific types of charges, in combination with the specific arrangement of the fermentation enhancement device, in order to increase growth of a fermenting microorganism and to suppress contaminants, such as bacteriophages and/or unwanted fungi and/or bacteria, through formation of Reactive Oxygen Species (ROS) and associated redox reactions.

Piezoelectric materials produce charges when exposed or placed under mechanical stress, which can come from various actuation sources such as a transducer (e.g. a piezoelectric transducer), an agitator, or a shaker. Thus, the fermentation enhancement device is arranged such that it can, during use, be coupled with an actuation source in order to actuate the virucidal layer and the proliferation layer of the electrical charge structure.

Examples of the presently disclosed fermentation enhancement device can be seen in the appended figures. For example, FIG. 1 shows an example of the presently disclosed fermentation enhancement device having a cylindrical shape, which is arranged for containing bacteria during a fermentation process.

The manufacturing of fermented products requires inoculation of selected fermenting microorganism, such as of the selected bacterial cells such as gram-negative bacteria, or gram-positive bacteria. In embodiments, the presently disclosed fermentation enhancement device is arranged for fermentation of a bacteria, fungi or algae that have an extracellular electron transfer mechanism (EET), which has been shown to be affected by electrical charges. The bacteria used may for example be Lactic Acid Bacteria (LAB).

In addition, the presently disclosed fermentation enhancement device may further be arranged to block passage of pathogens across the electrical charge structure, while allowing passage of nutrients and/or medium. The presently disclosed fermentation enhancement device may for example have a virucidal layer with a pore size of up to 20 nm, thereby acting as a physical barrier to protect the fermenting microorganism, for example to protect the LAB.

The presently disclosed fermentation enhancement device may have a proliferation layer with a porosity between 10-20 μm in size. As established, this pore size allows for a mass transfer such as the transfer of nutrients and waste biological products across the electrical charge structure of the fermentation enhancement device.

Evidently, the present disclosed fermentation enhancement device is not limited to any particular fermentation product, because it can be applied to any fermentation process, for example, in embodiments the fermentation enhancement device is used to ferment cow's milk to produce e.g. cheese or yogurt, but it may be applied to any other milk. The presently disclosed fermentation enhancement device may also be used to ferment other products such as fruits, meats, vegetables, and grains to produce fermented products, and alcoholic beverages.

In a further aspect, the present disclosure relates to a fermentation system comprising:
one or more fermentation enhancement device(s) as disclosed elsewhere herein;
one or more actuation source(s) arranged to actuate the electrical charge structure(s) of the fermentation enhancement device(s); and
a fermentation vessel accommodating the fermentation enhancement device(s).

Multiple fermentation enhancement devices may be used, and arranged in for example an array inside the fermentation vessel. During use, the fermentation enhancement device, or plurality of fermentation enhancement devices, are typically at least partially submerged in a medium/starting material, for example milk.

As described above a piezoelectric material's functionality relies on the application of mechanical stress. The vibration source is arranged to actuate the electrical charge structure, by being in contact, for example by being in direct contact with the electrical charge structures of the fermentation enhancement devices (FIG. 2).

In yet a further aspect, the present disclosure relates to a method for production of a fermented product, said method comprising:
adding, to a fermentation system as disclosed elsewhere herein, a starter culture of a fermenting microorganism and a suitable culture medium;
operating the fermentation system, including actuation of the actuation source such that electrical charges are generated at the electrical charge structure comprising an outer charge generated at the virucidal layer that kills and/or inactivates viruses and/or microorganisms, and an inner electrical charge generated at the proliferation layer that stimulates growth of the fermenting microorganism.

In even yet a further aspect, the present disclosure relates to method for fabricating a fermentation enhancement device as disclosed elsewhere herein, the method comprising:
a) Providing proliferation layer reagents comprising a piezoelectric material;
b) Mixing the proliferation layer reagents and fabricating the proliferation layer;
c) Providing virucidal layer reagents comprising a piezoelectric material;
d) Mixing the virucidal layer reagents and fabricating the virucidal layer;
e) Heat-treating the proliferation layer;
f) Heat-treating the virucidal layer;
g) Sintering the proliferation layer;
h) Sintering the virucidal layer;
i) binding the proliferation layer with the virucidal layer to form an electrical charge structure that is arranged such that actuation of said structure, by an actuation source, generates an electrical charge at the virucidal layer that kills and/or inactivates viruses and/or microorganisms, and an electrical charge at the proliferation layer that stimulates growth of a fermenting microorganism;
thereby obtaining a fermentation enhancement device as disclosed elsewhere herein.

### DESCRIPTION OF DRAWINGS

FIG. 1a is a cross-sectional diagrammatic frontal view of one embodiment of the presently disclosed fermentation enhancement device.

FIG. 1b is another cross-sectional diagrammatic view of one embodiment of the presently disclosed fermentation enhancement device.

FIG. 1c is a cross-sectional diagrammatic axial view of one embodiment of the presently disclosed fermentation enhancement device.

FIG. 2 is a diagrammatic view of one embodiment of the presently disclosed fermentation system comprising a plurality of fermentation enhancement devices.

FIG. 3a shows experimental results from using a fermentation system according to one embodiment of the present disclosure, for fermentation of lactic acid bacteria (LAB), in particular for 6 Hz.

FIG. 3b shows experimental results from using a fermentation system according to one embodiment of the present disclosure, for fermentation of lactic acid bacteria (LAB), in particular for 14 Hz.

FIG. 3c shows experimental results from using a fermentation system according to one embodiment of the present disclosure, for fermentation of lactic acid bacteria (LAB), in particular for 6 Hz and 16 Hz.

### DETAILED DESCRIPTION

The term “charge” as used herein refers to the electric charges accumulated on the surface of the piezoelectric material as well as close to it, in response to applied mechanical stress, for example, vibration.

The term “coefficient dᵢⱼ” or piezoelectric charge coefficient or piezoelectric constant, refers to a polarization generated per unit of mechanical stress applied to a piezoelectric material or, alternatively, it is the mechanical strain experienced by a piezoelectric material per unit of the electric field applied. The first subscript to d, “i” indicates the direction of polarization generated in the material when the electric field is zero or, alternatively, is the direction of the applied field strength and can be 1, 2 or 3. The second subscript to d, “j” is the direction of the applied stress or the induced strain and can be 1, 2, 3, 4, 5 or 6, respectively. In the case of d₃₃, both the applied stress and resultant polarization align along the vertical axis. It can be measured by using the Berlincourt method, laser interferometry, micro-displacement sensing and voltage piezo test. The piezoelectric constant is normally measured at room temperature and atmospheric pressure.

The term “opening” refers to an aperture, a gap, or an orifice that allows access or passage of a liquid, for example, a fermentation medium, a bacterial culture or starter culture, nutrients in liquid or powder form, or any other component that may be needed for fermentation into the fermentation enhancement device.

The term “porosity” refers to a measure of the void or empty spaces in a layer and/or in a piezoelectric material used in the present disclosure. It is indicated as a fraction of the volume of voids/empty spaces over the total volume for example as a value between 0 and 1 or as a percentage between 0% and 100%. There are several ways to measure or test porosity in a component for example Hg-porosimetry, He-pycnometry and electron microscopy.

Extracellular electron transfer (EET) processes are two way processes-transfer of electrons from inside the cell to outer electron acceptor, known as electrogenic microorganisms and transfer of electrons from outer electron source to the cell, known as electrotrophic microorganisms. The source of electron acceptor or sources can be metals, minerals or electrodes that are in contact with cell membranes (Singh and Kumar, 2022). Some well-known electrogenic bacteria, archaea, fungi (especially yeasts) and algae are listed in Tables 1, 2 and 3 in Thapa et al. 2022, and Table 1 of Garbini et al. 2023; Chapter 27, authored Singh and Kumar, of the book Microbial Biodegradation and Bioremediation (Second Edition) 2022 also describes EET in microorganisms.

A “fermenting microorganism” as defined herein can be any microorganism suitable for use in a desired fermentation process for the production of bio-based products. Suitable fermenting microorganisms include, without limitation, filamentous fungi, yeast, bacteria and algae.

The presently disclosed fermentation enhancement device provides a reliable, simple, and affordable solution to improve the efficiency of fermentation processes by increasing the production yield of a fermenting microorganism, such as bacteria, and killing phages and/or microorganisms, thus preventing failed fermentations. The fermentation enhancement device of the present disclosure may be used in a fermentation system, thus the present disclosure also relates to a fermentation system comprising one or more fermentation enhancement devices described herein, one or more actuation sources and a fermentation vessel.

The present disclosure also relates to methods for producing a fermented product, said methods comprising using a fermentation system as disclosed elsewhere herein.

One aspect of the present disclosure relates to a fermentation enhancement device for use in a bioreactor. The fermentation enhancement device may comprise an electrical charge structure defining an inner volume arranged for accommodating a fermentation process. The electrical charge structure typically comprises a piezoelectric proliferation layer exposed to the inner volume, and/or a piezoelectric virucidal layer arranged at an outer surface of the electrical charge structure. The fermentation enhancement device may comprise an opening configured to allow therethrough passage of fluids, such as nutrients, and/or fermenting microorganisms, such as bacteria. The fermentation enhancement device may be arranged such that actuation of the electrical charge structure, by an actuation source, generates an electrical charge at the virucidal layer that kills and/or inactivates viruses and/or microorganisms, for example via formation of ROS and associated redox reactions, and an electrical charge at the proliferation layer that stimulates growth of the fermenting microorganism.

The fermentation enhancement device of the present disclosure is typically arranged such that it generates, during use, upon actuation of an actuation source connected to the electrical charge structure, electrical charges at the proliferation layer stimulating growth of a fermenting microorganism, for example of bacteria, and electrical charges at the virucidal layer having a virucidal and/or fungicidal and/or bactericidal effect through generation of ROS and associated redox reactions.

The cross section of the electrical charge structure, along its axial length, such as from a first end to a second end, may have a certain shape, for example, the cross section may be a polygonal, such as a triangle, a square, a rectangle, a hexagonal, or a pentagonal, alternatively, the cross section may be rounded such as oval or circular. Any other shape that a person of skills in the art may find suitable, can also be used.

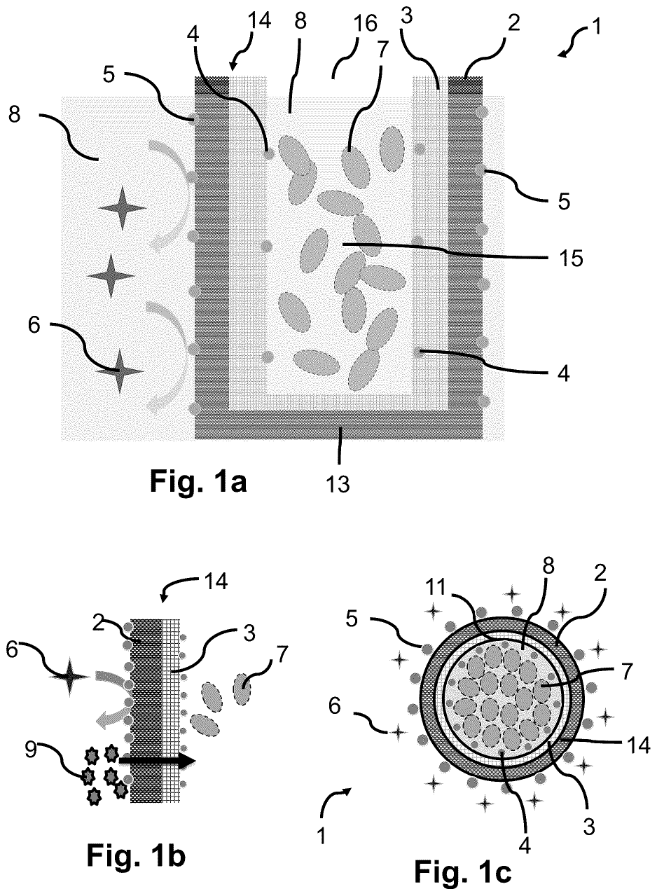

In one embodiment of the present disclosure the electrical charge structure has an elongated structure. The electrical charge structure may for example be a cylindrical structure. Further, the electrical charge structure may be a container. In a specific example, the electrical charge structure is a cylindrical container, as for example shown in FIG. 1, wherein the electrical charge structure defines an inner volume that is arranged for accommodating a fermentation process.

In specific example, the cross section of the electrical charge structure, along its axial length, such as from a first end to a second end, is rounded, such as circular, or a polygonal, such as rectangular or squared.

In specific example, the electrical charge structure is formed entirely from the proliferation layer and the virucidal layer. Thus, said layers may in such a case, be arranged into a container to accommodate a fermentation process in an inner volume thereof. As such, a first end of the electrical charge structure may be covered by the proliferation layer and/or the virucidal layer.

Alternatively, the electrical charge structure may comprise further layers, such as a support layer. The support layer may for example be arranged to seal off an opening of the other parts of the electrical charge structure (e.g. the proliferation layer and the virucidal layer). The proliferation layer and the virucidal layer may for example be provided as a tubular structure, and the support structure may thus be arranged to cover, such as to seal, a first end of said tubular structure, in order to form a container for accommodating the fermentation process.

In one embodiment of the present disclosure, the virucidal layer is arranged at an outer surface of the electrical charge structure and the proliferation layer is arranged on an inner surface of the electrical charge structure. For example, an inner surface of the electrical charge structure may be made of the proliferation layer, and an outer surface of the electrical charge structure may be made of the virucidal layer.

In one embodiment of the present disclosure the piezoelectric proliferation layer, or a part thereof, comprises a piezoelectric material. The electrical charge structure may have an inner surface that is, partly or fully, covered by the proliferation layer.

The proliferation layer may be in some embodiments constitute, at least a part of, an inner layer of the electrical charge structure or the innermost layer of the electrical charge structure and it may be configured to increase growth of the fermenting microorganism, enhance metabolism of the fermenting microorganism, and promote the production of metabolites of the fermenting microorganism, such as upon actuation of the layer by an actuation source.

In one embodiment of the present disclosure the piezoelectric virucidal layer, or a part thereof, comprises a piezoelectric material. The electrical charge structure may have an outer surface that is, partly or fully, covered by the virucidal layer. The virucidal layer may, in some embodiments of the present disclosure, constitute, at least a part of, an outer layer of the electrical charge structure or even the outermost layer of the electrical charge structure, and it may be configured to stop, destroy and/or inactivate viruses, such as bacteriophages, and/or microorganisms, such as upon actuation of the layer by an actuation source.

In one embodiment of the present disclosure, the proliferation layer is arranged to abut the virucidal layer. In another example, the proliferation layer may be covered by the virucidal layer. In other examples, the proliferation layer and the virucidal layer are separated, at least in part, by a further layer, such as an electrode layer, comprising a conducting material.

In one embodiment of the present disclosure, the electrical charge structure comprises the opening, and it is arranged to allow nutrients, media, and/or fermenting microorganisms, such as bacteria, to flow in and out of the inner volume of the electrical charge structure. The flow rate may depend on the size of the fermentation enhancement device and/or the corresponding system.

Potentially, the flow rate may be 0. In embodiments, in specific examples, no upper limit to the flow rate may exist, however flow rates that may be harmful to the fermenting microorganism, such as the bacteria and/or the device is avoided.

In embodiments, during fermentation, if the fermenting microorganism already have nutrients, the flow rate may be minimal (such as on the order of 1 μL/min) or even zero, but if the fermentation enhancement device(s) is previously empty, it is arranged such that it may be filled within the first minutes of culturing. Thus, the flow rate during filling may depend on the size of the reactor. The maximum flow rate may for example be around 100 mL/min

In other examples, during fermentation, the flow rate may be in the range of from 0 to 100 mL/min. In embodiments, the flow rate is such that it allows for sufficient exchange of nutrients, gasses, and/or waste products.

In embodiments, the flow may for example be at least 0 μL/min, for example between 0 μL/min and 100 mL/min, or between 1 μL/min to 1 mL/min. The flow rate may for example depend on the device design, such as the size of the device and/or the requirements of the fermenting microorganism for efficient culturing.

The flow may for example be driven by diffusion and/or convection, such as a forced convective flow, which may be driven by a convective flow, such as a stirrer or a pump.

In one embodiment of the present disclosure, a second end of the electrical charge structure comprises the opening. The second end is typically arranged to be positioned upwardly during use.

In specific examples of the present disclosure, the electrical charge structure comprises multiple openings, such as a top opening, and one or more side openings.

In one embodiment of the present disclosure, a first end, opposite to the second end, of the electrical charge structure comprises an opening or is covered by a substrate.

In one embodiment of the present disclosure, when the fermentation enhancement device is being used there may be a flow of nutrients, media, and/or fermenting microorganisms through the opening. For example, the flow may vary or be constant while the fermentation enhancement device is being used.

In one embodiment of the present disclosure, when the fermentation enhancement device is being used, there may be no flow of nutrients, media, and/or fermenting microorganism through the opening.

In general, a smaller inner dimension of the electrical charge structure (and thereby the dimension of the inner volume of the electrical charge structure), leads to a larger surface to volume ratio (i.e. between the exposed area of the proliferation layer and the inner volume), and thereby, an increased amount of interaction between the fermenting microorganism and the proliferation layer, which may be desirable for certain applications.

In one embodiment of the present disclosure, the electrical charge structure device is cylindrical with an inner diameter of at least 0.5 mm, such as an inner diameter of at least 0.7 mm, such as an inner diameter of at least 1 mm, such as an inner diameter of at least 1.3 mm, such as an inner diameter of at least 1.5 mm, such as an inner diameter of at least 1.7 mm, such as an inner diameter of at least 2 mm, such as an inner diameter of at least 2.3 mm, such as an inner diameter of at least 2.5 mm, such as an inner diameter of at least 2.7 mm, such as an inner diameter of at least 3 mm, such as an inner diameter of at least 3.2 mm, such as an inner diameter of at least 3.5 mm, such as an inner diameter of at least 3.7 mm, such as an inner diameter of at least 4 mm, such as an inner diameter of at least 4.2 mm, such as an inner diameter of at least 4.5 mm, such as an inner diameter of at least 4.7 mm, such as of an inner diameter between 5 and 7 mm, such as of an inner between 6 and 9 mm, such as of an inner between 8 and 11 mm, such as of an inner between 10 and 13 mm, such as of an inner between 12 and 15 mm, such as of an inner between 14 and 17 mm, such as of an inner between 16 and 19 mm, such as of an inner of at the most 20 mm, such as of an inner of at the most 15 mm, such as of an inner of at the most 10 mm.

However, typically the electrical charge structure has an inner diameter of at least 0.5 mm and at the most 20 mm, irrespective of the cross sectional shape.

In one embodiment of the present disclosure the thickness of the electrical charge structure of the fermentation enhancement device is 0.005 mm or more, such as at least 0.01 mm, such as at least 0.05 mm, such as at least 0.1 mm, such as at least 0.02 mm, such as at least 0.5 mm, and at the most 1 mm, such as at the most 1.5 mm, such as at the most 2 mm, such as at the most 2.5 mm, such as at the most 3 mm, such as at the most 3.5 mm, such as at the most 4 mm, such as at the most 4.5 mm, such as at the most 5 mm, such as at the most 5.5 mm, such as at the most 6.5 mm, such as at the most 7.5 mm, such as at the most 8.5 mm, such as at the most 9.5 mm, such as at the most 10 mm, such as at the most 11 mm, such as at the most 12 mm, such as at the most 13 mm, such as at the most 14 mm, such as at the most 15 mm, such as at the most 16 mm, such as at the most 17 mm, such as at the most 18 mm, such as at the most 19 mm, such as at the most 20 mm.

In one embodiment of the present disclosure the proliferation layer is thicker than the virucidal layer.

In one embodiment of the present disclosure the virucidal layer has a thickness of 1 nm or more, such as of 5 nm or more, such as of 10 nm or more, such as of 50 nm or more.

In one embodiment of the present disclosure the virucidal layer is thicker than the proliferation layer. For example, in one embodiment of the present disclosure the proliferation layer has a thickness of 1 nm or more, such as of 5 nm or more, such as of 10 nm or more, such as of 50 nm or more.

In one embodiment of the present disclosure the proliferation layer is about as thick as the virucidal layer.

A piezoelectric material is a material that produces an electric charge when exposed or placed under mechanical stress or pressure. Piezoelectric materials also exhibit mechanical strain when an electrical field is applied.

There are several materials that are known to posses piezoelectric properties, as found e.g. in Liu et al. 2018 and Safaei et al. 2019, for example hydroxyapatite (poled ceramic), crystals such as quartz, topaz, Rochelle salt, schorl tourmaline; polymers such as PVDF,P(VDF-TrFE), ceramics such as lead zirconate titanate (PZT), lead titanate, lead niobate, barium strontium titanate, BiB₃O₆, BiFeO₃, Al₃AsO₇, Al₃PO₇, Ga₃AsO₇, GaPO₇, ZnO, AlN, barium titanate (Single Crystal), CBT, NBBT95/5, Mn—KNN, BZT-BCT, GaPBi₃, GeSe, SnS, SnSe, GeS, ZnO nanorods, PZT nanoshell, GaN Nanowires, barium titanate piezoelectrics are (BaTiO₃), potassium sodium niobate (KNaNbO₃), barium calcium zirconium titanate (BaCaZrTiO₃), lithium niobium oxide (LiNbO₃), sodium niobium oxide (NaNbO₃) or potassium niobium oxide (KNbO₃).

In one embodiment of the present disclosure the piezoelectric proliferation layer, or a part thereof, comprises a piezoelectric material. Similarly, the piezoelectric virucidal layer, or a part thereof, may comprise a piezoelectric material.

In specific examples, the piezoelectric proliferation layer and the piezoelectric virucidal layer comprise different piezoelectric materials. Alternatively, the piezoelectric materials of the two layers may be the same.

It may be desirable that the proliferation layer and the virucidal layer have different chemical and/or physical properties, such as a different porosity, and/or different piezoelectric coefficient (dᵢⱼ).

The piezoelectric coefficient d (also known as the piezoelectric modulus or the piezoelectric charge constant) quantifies the polarization of the piezoelectric material per unit of mechanical stress. As a result, the piezoelectric coefficient is typically given in Coulombs per Newton.

In general, piezoelectricity is described by a tensor of coefficients (dᵢⱼ). The first subscript to d indicates the direction of the charge motion associated with the applied stress. The second subscript is the direction of the applied stress.

Specifically, d₃₃ describes the ability of a piezoelectric element to induce a polarization in the 3-axis (parallel to direction in which the ceramic element is polarized) per unit stress applied in the same direction.

Thus, in one embodiment of the present disclosure the proliferation layer comprises a piezoelectric material with a piezoelectric coefficient (dᵢⱼ), such as d₃₃, of at least 25 pC/N, such as of 35-45 pC/N, such as of 40-55 pC/N, such as of 50-65 pC/N, such as of 60-75 pC/N, such as of 70-85 pC/N, such as of 80-95 pC/N, such as of 90-105 pC/N, such as of 100-115 pC/N, such as of at the most 120 pC/N.

In a further embodiment of the present disclosure the proliferation layer comprises a piezoelectric material with a piezoelectric coefficient (d₃₃) of at least 25 pC/N, such as of 35-45 pC/N, such as of 40-55 pC/N, such as of 50-65 pC/N, such as of 60-75 pC/N, such as of 70-85 pC/N, such as of 80-95 pC/N, such as of 90-105 pC/N, such as of 100-115 pC/N, such as of at the most 120 pC/N. Thus, piezoelectric materials having a d₃₃ of at least 25 pC/N may be suitable for being the main constituent of the proliferation layer.

In one embodiment of the present disclosure the virucidal layer comprises a piezoelectric material with a piezoelectric coefficient (di;), such as d₃₃, greater than 150 pC/N, such as of 165-175 pC/N, such as of 160-170 pC/N, such as of 175-185 pC/N, such as of 170-180 pC/N, such as of 185-195 pC/N, such as of 180-190 pC/N, such as of at the most 200 pC/N.

In one embodiment of the present disclosure the virucidal layer comprises a piezoelectric material with a piezoelectric coefficient (d₃₃) greater than 150 pC/N, such as of 165-175 pC/N, such as of 160-170 pC/N, such as of 175-185 pC/N, such as of 170-180 pC/N, such as of 185-195 pC/N, such as of 180-190 pC/N, such as of at the most 200 pC/N. Thus, any piezoelectric material having a d₃₃ greater than 150 pC/N is suitable for being the main constituent of the virucidal layer.

A person of skill in the art would know which piezoelectric material is suitable for the proliferation layer and/or for the virucidal layer based on the properties of the materials and the desired dᵢⱼ, such as the desired d₃₃.

In one embodiment of the present disclosure the fermentation enhancement device may comprise the same piezoelectric material but differ in terms of other properties such as piezoelectric coefficient and/or porosity. For example, the proliferation layer may comprise slightly poled BaTiO₃ and the virucidal layer may comprise highly poled BaTiO₃.

In an embodiment of the present disclosure, the fermentation enhancement device may further be arranged to block passage of pathogens across the electrical charge structure, while allowing passage of nutrients and/or medium. The presently disclosed fermentation enhancement device may for example have a virucidal layer with a pore size of up to 20 nm, thereby acting as a physical barrier to protect the LAB. Hence, the electrical charge structure may be arranged to allow passage of nutrients and/or medium across the walls of said structure. For example, by comprising porous structures, for example a porous proliferation layer and a porous virucidal layer and/or porous further layers for example a porous electrode layer.

In one embodiment of the present disclosure the proliferation layer may comprise pores having a size of at the most 100 μm, such as a size of 5 to 15 μm, such as of 10 to 20 μm, such as of 11 to 21 μm, such as of 12 to 22 μm, such as of 13 to 23 μm, such as of 14 to 24 μm, such as of 9 to 19 μm, such as of 10 to 18 μm, such as of 8 to 17 μm, such as of 10 to 16 μm, such as of 15 to 25 μm, such as of 20 to 30 μm, such as of 25 to 35 μm, such as of 30 to 40 μm, such as of 35 to 45 μm, such as of 40 to 50 μm, such as of 45 to 55 μm, such as of 60 to 70 μm, such as of 65 to 75 μm, such as of 70 to 80 μm, such as of 75 to 85 μm, such as of 80 to 90 μm, such as of 85 to 95 μm, or 10 to 20 μm in size.

A porous electrical charge structure (i.e. at least part of said structure) allows for mass transfer across the structure, such as the transfer of nutrients and waste biological products across the electrical charge structure of the fermentation enhancement device.

For example, nutrients may move from the outside environment into the fermentation enhancement device and the wasted biological products may move from the inside to the outside of the fermentation enhancement device, through for example diffusion, or convective flow.

Also, the porosity volume and pore size of the fermentation enhancement device may provide a larger surface area which may facilitate the improved interaction between the fermenting microorganism and the proliferation layer, thus acting to further increase the growth.

In one embodiment of the present disclosure, the total porosity in the proliferation layer may be of at least 5% by volume and at the most 70% by volume, at least 30% by volume, such as about 35% by volume. For example, the total porosity in the proliferation layer may be of at least 10% by volume, such as of at least 12% by volume, such as of at least 15% by volume, such as of at least 17% by volume, such as of at least 20% by volume, such as of at least 23% by volume, such as of at least 25% by volume, such as of at least 27% by volume, such as of at least 32% by volume.

In one embodiment of the present disclosure, the virucidal layer has pores of a size of 5 to 100 nm, such as of 5 to 15 nm, such as of 10 to 25 nm, such as of 20 to 30 nm, such as of 25 to 35 nm, such as of 30 to 40 nm, such as of 35 to 45 nm, such as of 40 to 50 nm, such as of 45 to 55 nm, such as of 50 to 60 nm, such as of 60 to 70 nm, such as of 65 to 75 nm, such as of 70 to 80 nm, such as of 80 to 90 nm, such as 85 to 95 nm, such as of 15 nm or less, such as of 6 to 15 nm, such as of 7 to 15 nm, such as of 8 to 15 nm, such as of 9 to 15 nm, such as of 10 to 15 nm, such as of 5 to 11 nm, such as of 5 to 12 nm, such as of 5 to 13 nm, such as of 5 to 14 nm.

In one embodiment of the present disclosure, the total porosity of the virucidal layer has a total porosity of at least 5% by volume and at the most 70% by volume, such as at least 20% by volume, such as about 30% by volume, such as 10 to 70% by volume, such as 15 to 70% by volume, such as 20 to 70% by volume, such as 25 to 70% by volume, such as 30 to 70% by volume, such as 35 to 70% by volume, such as 40 to 70% by volume, such as 10 to 65% by volume, such as 10 to 60% by volume, such as 10 to 55% by volume, such as 10 to 50% by volume, such as 10 to 45% by volume, such as 10 to 40% by volume.

In one embodiment of the present disclosure, the total porosity may be kept to the minimum possible so that the highest possible dᵢⱼ can be obtained.

The pore size range in the outer layer may function as a filter by preventing the entry of the pathogens (up to 20 nm) from the medium into the fermentation enhancement device, even in the case that, during use, the virucidal layer does not generate electrical charge to kill/inactivate viruses or microorganisms, the small pore sizes will function as a physical barrier to block pathogens, such as bacteriophages, to protect the fermenting microorganism, while at the same time allowing passage of medium and/or nutrients.

However, In other embodiments of the present disclosure both the proliferation layer and the virucidal layer are non-porous. Alternatively, the proliferation layer may be porous while at the same time the virucidal layer may be non-porous, or vice versa, wherein the porous layer(s) may be as specified above.

In one embodiment of the present disclosure, the fermentation enhancement device is arranged such that the charge generated, during use, at the proliferation layer is different from the charge at the virucidal layer. Typically, the electrical charge generated at the virucidal layer is higher than the electrical charge generated at the proliferation layer. Thus, the electrical charge generated at the proliferation layer may be lower than the electrical charge generated at the virucidal layer.

The electrical charges (i.e. the inner charge generated at the proliferation layer and the outer charge generated at the virucidal layer), as a result of actuation by an actuation source may depend on the piezoelectric constants of the layers and the stresses applied to the layers. Thus, the charge density of the proliferation layer and the virucidal layer may be a result of the piezoelectric constants of the layers, the sizes of the layers and the stresses applied to the layers.

Typically, the resulting charge density, upon application of a mechanical stress to a material (e.g. the proliferation layer and/or the virucidal layer) is given by q_{d}=d*F/A Where q_{d} is the charge density given in Coulombs per square meter, d is the piezoelectric constant given in Coulombs per Newton, F is the force applied in Newtons, and A is the area in square meters. Commonly, the piezoelectric constant is given as d₃₃, and the force is thus applied in the corresponding direction (i.e. direction 3 which is parallel to direction in which the ceramic element is polarized).

As an example, for a piezoelectric layer having an area of 0,000314 m (i.e. a disk with 1 cm radius), a piezoelectric constant d₃₃ of 190 pC/N, and wherein the applied force amounts to 1 N, the resulting charge density generated is in the range of

$$6.05 \cdot {10}^{- 7} \frac{C}{{m}^{2}} .$$

Thus, the resulting electrical charge density is proportional to the piezoelectric constant of the material. In this regard, it should be noted that the proliferation layer and the virucidal layers may be of the same material, even in the specific case wherein said layers have different piezoelectric constants. For example, the proliferation layer and the virucidal layer may be of the same material, but may have been poled differently, in order to obtain layers of different piezoelectric constants. For example a piezoelectric constant of the virucidal layer that is higher than the piezoelectric constant of the proliferation layer.

The piezoelectric constants and/or the fermentation enhancement device of the present disclosure may be arranged such that the inner charge (i.e. facing the inner volume) is so high as to stimulate bacterial growth, such as growth of the fermenting microorganism, while not giving rise to a virucidal effect and/or an effect that is harmful for the bacteria or other fermenting microorganism. At the same time, the piezoelectric constants and/or the fermentation enhancement device of the present disclosure may be arranged such that the outer charge is higher than the inner charge, and wherein the outer charge is sufficiently high to give rise to a virucidal and/or fungicidal and/or bactericidal effect through formation of ROS and associated redox reactions.

In one embodiment of the present disclosure, the fermentation enhancement device is arranged such that actuation of the electrical charge structure generates a charge at the proliferation layer, and wherein said charge stimulates growth of fermenting microorganisms.

In one embodiment of the present disclosure, the fermentation enhancement device is arranged such that actuation of the electrical charge structure generates a charge at the virucidal layer that generates reactive oxygen species (ROS) and associated redox reactions.

In one embodiment of the present disclosure, the charge generated at the virucidal layer kills and/or inactivates bacteriophages.

In one embodiment of the present disclosure, the charge generated at the virucidal layer kills and/or inactivates prokaryotic microorganisms.

In one embodiment of the present disclosure, the charge generated at the virucidal layer kills and/or inactivates eukaryotic microorganisms.

In one embodiment of the present disclosure, the prokaryotic microorganisms are bacteria and/or archaea.

In one embodiment of the present disclosure, the charge generated at the eukaryotic microorganisms are algae and/or fungi.

In some embodiments, the charge generated at the proliferation layer is different from the charge generated at the virucidal layer. This may be due to the different properties of the materials used in the proliferation layer and virucidal layer, and in particular to their different piezoelectric strength (or piezoelectric constant).

Because of the different charge at the proliferation layer compared to the charge at the virucidal layer, certain microorganisms may proliferate when exposed to the proliferation layer but not when exposed to the virucidal layer, where ROS and associated redox reactions are generated.

In one embodiment of the present disclosure, the fermentation enhancement device comprises an actuation source arranged for actuation of the electrical charge structure (e.g. of the proliferation layer and/or of the virucidal layer) or alternatively multiple electrical charge structure, in case the fermentation enhancement device comprises more than one. The fermentation enhancement device may also comprise a fermentation vessel arranged for accommodating one or more electrical charge structures.

In one embodiment of the present disclosure, the fermentation enhancement device is suitable for culturing bacteria, such as gram-negative bacteria, or gram-positive bacteria, such as bacteria having extracellular electron transfer mechanism (EET). In embodiments, LAB.

In one embodiment of the present disclosure, the fermentation enhancement device is suitable for culturing eukaryotic microorganisms, such as fungi or algae, such as eukaryotic microorganisms having extracellular electron transfer mechanism (EET).

In one embodiment of the present disclosure, the fermentation enhancement device is arranged for being contained by a bioreactor.

In a further aspect, the present disclosure relates to a fermentation system comprising: one or more fermentation enhancement device(s) as disclosed elsewhere herein. In embodiments, the fermentation system comprises one or more actuation source(s) arranged to actuate the electrical charge structure(s) of the fermentation enhancement device(s). Typically each fermentation system comprises a single fermentation structure. In some embodiments, the fermentation system comprises a fermentation vessel accommodating the fermentation enhancement device(s). The fermentation vessel may desirably be arranged to contain a culture medium, and further, be arranged such that, during use, at least a part of the electrical structure(s) are submerged in the culture medium.

In one specific embodiment the fermentation system is a bioreactor. As such, the fermentation system may be a system that supports a biologically active environment.

Further, the system may be arranged to provide a controlled environmental conditions inside the fermentation vessel, such as a predetermined temperature, a predetermined nutrient concentration, a predetermined pH, and a predetermined type and concentration of dissolved gasses (e.g. oxygen for aerobic fermentations).

In such a case it may be desirable if the fermentation vessel is arranged to form a sealed environment that contains at least the electrical structure(s). The actuation source(s) may be provided in said sealed environment, or provided outside with a physical connection to the electrical charge structure(s), such that said source(s) are capable of actuation the electrical charge structure(s).

In order to generate electrical charges at the electrical charge structure, the fermentation system may need to be actuated, such as by mechanical stress and/or vibrations, which may be provided by various sources. In one embodiment of the present disclosure, the actuation source is a transducer, such as a piezoelectric transducer, an agitator and/or a shaker.

The fermentation enhancement device(s) may be detachable from the fermentation system, for example such that the rest of the fermentation system can be reused with one or more new fermentation enhancement devices. The fermentation enhancement device(s) may for example be adhered to the substrate or directly to the actuation source, in order to form the fermentation system. Thereafter, the fermentation enhancement device(s) may be detached and replaced with a new set of fermentation enhancement devices.

In one embodiment of the present disclosure, the fermentation system comprises a plurality of fermentation enhancement devices. Such a fermentation system may comprise one or more actuation sources. Thus, one actuation source may be arranged to actuate a plurality of fermentation enhancement devices/electrical charge structures, alternatively or additionally, multiple electrical charge structures may each be arranged to be actuated by a single, different, actuation source.

In one embodiment of the present disclosure, the fermentation system comprises a plurality of actuation sources, such as wherein each fermentation enhancement device is arranged to be actuated by a different actuation source. In another embodiment of the present disclosure, a plurality of fermentation enhancement devices are arranged to be actuated by a single actuation source.

In an embodiment of the present disclosure, an actuation source is in physical contact, e.g. direct physical contact, with the electrical charge structure, such as wherein the actuation source abuts the electrical charge structure(s).

In one embodiment of the present disclosure, the actuation source is arranged to provide a vibration force of between 0.1 and 20 N, such as of 0.1 to 18 N, such as of 0.1 to 16 N, such as of 0.1 to 14 N, such as of 0.1 to 12 N, such as of 0.1 to 10 N, such as of 0.1 to 8 N, such as of 0.3 to 18 N, such as of 0.5 to 18 N, such as of 0.8 to 18 N, such as of 1 to 18 N, such as of 2 to 18 N, such as of 3 to 18 N, such as of 4 to 18 N, such as of 5 to 18 N, such as of 6 to 18 N, such as of 7 to 18 N, such as of 8 to 18 N, such as of 1 to 4 N, at a low frequency, such as 5 to 20 Hz.

In one embodiment of the present disclosure, the actuation source(s) are arranged to provide a vibration force of between 0.1-20 N, such as between 1-4 N, at low frequency, such as 5-20 Hz.

In one embodiment of the present disclosure, two or more fermentation enhancement devices are positioned on a same horizontal plane inside the fermentation vessel. Such fermentation enhancement devices may be arranged to be actuated by the same vibration source. As such, the horizontal plane may be defined by the vibration source, or a support structure to which the actuation source is coupled.

In one embodiment of the present disclosure, two or more fermentation enhancement devices are positioned on different horizontal planes inside the fermentation vessel. Such fermentation enhancement devices may be arranged such that the same vibration source actuates fermentation enhancement devices on the same horizontal plane. As such, each horizontal plane may be defined by a different vibration source, or a different support structure to which an actuation source is coupled (the same single actuation source or different actuation sources).

In some embodiments, two or more fermentation enhancement devices, such as an array of fermentation enhancement devices may be arranged in the fermentation vessel.

In one embodiment of the present disclosure, the electrical charge structures of each fermentation enhancement device is separated by a distance from each other, such as wherein said distance is equal to or larger than the radius of the fermentation enhancement device.

In one embodiment of the present disclosure, the fermentation system is suitable for conducting a fermentation process based on the use of gram-negative bacteria or gram-positive bacteria, such as based on the use of bacteria having EET, as discussed elsewhere herein.

One aspect of the present disclosure relates to a method for production of a fermented product, said method comprising:
adding, to a fermentation system comprising one or more fermentation enhancement device(s) as disclosed elsewhere herein, a starter culture of a fermenting microorganism and a suitable culture medium;
operating the fermentation system, including actuation of the actuation source such that electrical charges are generated at the electrical charge structure comprising an outer charge generated at the virucidal layer that kills and/or inactivates viruses and/or microorganisms, and an inner electrical charge generated at the proliferation layer that stimulates growth of the fermenting microorganism, thereby producing the fermented product.

In some embodiments of the present disclosure, the electrical charge structures of one or more fermentation enhancement devices are at least partially submerged in the medium.

In some embodiments of the present disclosure, the electrical charge structures of one or more fermentation enhancement devices are submerged in the medium.

In some embodiments of the present disclosure, the step of operating the fermentation system comprises providing vibrations to one or more fermentation enhancement devices along a horizontal direction. Said step may alternatively or additionally comprise providing a suitable growth atmosphere inside the fermentation vessel.

In some embodiments of the present disclosure, the step of operating the fermentation system comprises generating an electrical charge at the proliferation layer that stimulates bacterial growth, such as growth of the fermenting microorganism.

In some embodiments of the present disclosure, the step of operating the fermentation system comprises generating an electrical charge at the virucidal layer that kills and/or inactivates viruses and/or microorganisms.

The manufacturing of fermented products requires the inoculation of selected fermenting microorganisms, which in some embodiments are selected bacterial cells. There are several fermenting microorganism that may be used in fermentation, such as bacteria, archaea, fungi, (for example yeasts) and algae. In one embodiment, the fermenting microorganism is a bacteria, for example gram-negative bacteria, or gram-positive bacteria.

In some embodiments of the present disclosure the fermenting microorganism used with the described fermentation enhancement device is a microorganism having extracellular electron transfer mechanism (EET) . . . . Both certain prokaryotic and eukaryotic microorganisms are known to have EET, as found in these scientific reviews and articles. Some well-known electrogenic bacteria, archaea, fungi (especially yeasts) and algae are listed in Tables 1, 2 and 3 in Thapa et al. 2022, and Table 1 of Garbini et al. 2023; Chapter 27, authored Singh and Kumar, of the book Microbial Biodegradation and Bioremediation (Second Edition) 2022 also describes EET in microorganisms.

For example, bacteria having EET are lactic acid bacteria, such as Lactococcus, Enterococcus, Streptococcus, Pediococcus, Leuconostoc, Oenococcus, Weissella, and group I and group Ill lactobacilli. Other bacteria having EET are also suitable for being used with a fermentation enhancement device of the present disclosure.

In some embodiments of the present disclosure, gram-positive bacteria are used with the described fermentation enhancement device.

In some embodiments of the present disclosure, gram-negative bacteria are used with the described fermentation enhancement device.

In some embodiments of the present disclosure, yeasts having EET are used with the described fermentation enhancement device.

In some embodiments of the present disclosure, algae having EET are used with the described fermentation enhancement device.

In one embodiment of the present disclosure, the fermentation enhancement device is used to produce dairy products. Such a process desirably uses LAB, which digests glucose to produce lactic acid, turning their medium or starting materials (e.g. milk) into yogurt, cheese, or butter. The amount of lactic acid produced per unit of glucose is typically called yield and describes how efficient the acidification of the milk is.

Lactic Acid Bacteria (LAB) are used for fermentation processes. They are a diverse group of aerotolerant, saccharolytic bacteria that mainly use fermentation for energy conservation.

In one embodiment of the present disclosure the culture medium or starting material used for the culture may be cow milk, however, several other starting materials could be used such as goat milk, sheep milk, yak milk, buffalo milk, mare milk, and camel milk, as well as others. As an alternative, the culture medium can be nutrient broth such as M17 or GM17. A person of skill in the art would know how to select a starting material and fermentation medium based on common general knowledge within the fermentation field.

In some embodiments of the present disclosure the fermentation enhancement device is used to ferment milk to produce e.g. cheese or yogurt, however, the fermentation enhancement device may be used to ferment fruits, meats, vegetables, and grains to produce fermented meat products, fermented fish, and other seafood products, fermented root, and tuber products, fermented legumes, fermented vegetables, alcoholic beverages and other fermented product e.g. vinegar, nata, pidan, maing, koji, puer tea, fuzhuan brick, kombucha, soy sauce, miso, doenzong, sake, Tempe. In one of the embodiments the fermentation enhancement device also may be used in the manufacture of chocolate, the cocoa bean fermentation is generally considered a processing aid.

The present disclosed fermentation enhancement device is not limited to any particular fermentation product because it can be applied to any fermentation process, for example, in embodiments the fermentation enhancement device is used to ferment milk (e.g. cow milk, goat milk, sheep milk, yak milk, buffalo milk, mare milk, camel milk) to produce e.g. cheese or yogurt, but in one of the embodiments the fermentation enhancement device is used to ferment fruits, meats, vegetables, and grains to produce fermented meat products, fermented fish and other seafood products, fermented root, and tuber products, fermented legumes, fermented vegetables, alcoholic beverages and other fermented product e.g. vinegar, nata, pidan, maing, koji, puer tea, fuzhuan brick, kombucha, soy sauce, miso, doenzong, sake, Tempe. In one embodiment of the present disclosure the fermentation enhancement device also may be used in the manufacture of chocolate, the cocoa bean fermentation is generally considered a processing aid.

Hence, in one embodiment of the present disclosure, it is disclosed a method for production of a fermented dairy product, said method comprising:
adding, to a fermentation system comprising one or more fermentation enhancement device(s) as disclosed elsewhere herein, a starter culture of a suitable fermenting microorganism and a suitable culture medium;
operating the fermentation system thereby producing the fermented diary product.

In one embodiment of the present disclosure, it is disclosed a method for production of a fermented fish product, said method comprising:
adding, to a fermentation system comprising one or more fermentation enhancement device(s) as disclosed elsewhere herein, a starter culture of a suitable fermenting microorganism and a suitable culture medium;
operating the fermentation system thereby producing the fermented fish product.

In one embodiment of the present disclosure, it is disclosed a method for production of a fermented meat product, said method comprising:
adding, to a fermentation system comprising one or more fermentation enhancement device(s) as disclosed elsewhere herein, a starter culture of a suitable fermenting microorganism and a suitable culture medium;
operating the fermentation system thereby producing the fermented meat product.

In one embodiment of the present disclosure, it is disclosed a method for production of a fermented vegetable product, said method comprising:
adding, to a fermentation system comprising one or more fermentation enhancement device(s) as disclosed elsewhere herein, a starter culture of a suitable fermenting microorganism and a suitable culture medium;
operating the fermentation system thereby producing the fermented vegetable product.

In one embodiment of the present disclosure, it is disclosed a method for production of a fermented legumes product, said method comprising:
adding, to a fermentation system comprising one or more fermentation enhancement device(s) as disclosed elsewhere herein, a starter culture of a suitable fermenting microorganism and a suitable culture medium;
operating the fermentation system thereby producing the fermented legumes product.

In one embodiment of the present disclosure, it is disclosed a method for production of a fermented staple food, said method comprising:
adding, to a fermentation system comprising one or more fermentation enhancement device(s) as disclosed elsewhere herein, a starter culture of a suitable fermenting microorganism and a suitable culture medium;
operating the fermentation system thereby producing the fermented staple food.

Examples of fermented staple foods are bread, are fermented cereal porridges.

In one embodiment of the present disclosure, it is disclosed a method for production of a fermented coffee product, said method comprising:
adding, to a fermentation system comprising one or more fermentation enhancement device(s) as disclosed elsewhere herein, a starter culture of a suitable fermenting microorganism and a suitable culture medium;
operating the fermentation system thereby producing the fermented coffee product.

In one embodiment of the present disclosure, it is disclosed a method for production of a fermented cocoa product, said method comprising:
adding, to a fermentation system comprising one or more fermentation enhancement device(s) as disclosed elsewhere herein, a starter culture of a suitable fermenting microorganism and a suitable culture medium;
operating the fermentation system thereby producing the fermented cocoa product.

In one embodiment of the present disclosure, it is disclosed a method for production of a fermented sauce, said method comprising:
adding, to a fermentation system comprising one or more fermentation enhancement device(s) as disclosed elsewhere herein, a starter culture of a suitable fermenting microorganism and a suitable culture medium;
operating the fermentation system thereby producing the fermented sauce.

Examples of fermented sauces are fermented vinegar and fermented soy sauce.

In one embodiment of the present disclosure, it is disclosed a method for production of a fermented beverage, said method comprising:
adding, to a fermentation system comprising one or more fermentation enhancement device(s) as disclosed elsewhere herein, a starter culture of a suitable fermenting microorganism and a suitable culture medium;
operating the fermentation system thereby producing the fermented beverage.

For example, the method disclosed herein may be used to produce a fermented alcoholic beverage.

One aspect of the present disclosure relates to a method for fabricating the fermentation enhancement device comprising the steps of:
a) Providing reagents for the proliferation layer, the reagents comprising a piezoelectric material;
b) Mixing the reagents and fabricating the proliferation layer;
c) Providing reagents for a virucidal layer, the reagents comprising a virucidal material;
d) Mixing the reagents and fabricating the virucidal layer;
e) Heat-treating the proliferation layer;
f) Heat-treating the virucidal layer;
g) Sintering the proliferation layer;
h) Sintering the virucidal layer;
i) Binding the proliferation layer with the virucidal layer to form an electrical charge structure that is arranged such that actuation of said structure, by an actuation source, generates an electrical charge at the virucidal layer that kills and/or inactivates viruses and/or microorganisms, and an electrical charge at the proliferation layer that stimulates growth of a fermenting microorganism,
thereby obtaining a fermentation enhancement device of the present disclosure.

Processes for fabricating the proliferating layer and virucidal layer are known in the art and a skilled person is capable of selecting a suitable one based on their common general knowledge.

In one embodiment of the present disclosure, the proliferation layer and/or the virucidal layer is fabricated by a miscellaneous process. Examples of miscellaneous processes are sacrificial templating, foam casting, chemical vapor deposition and its variants, physical vapor deposition and variants thereof.

In one embodiment of the present disclosure, the proliferation layer and/or the virucidal layer is fabricated by a precursor-based process. Examples of precursor-based processes are aerosol-gel coating, spin coating, ink-jet printing, dip-coating, gel-casting, additive manufacturing, and variants thereof.

In one embodiment of the present disclosure, the proliferation layer and/or the virucidal layer is fabricated by a particulate-based process. Examples of particulate-based processes are tape casting, screen printing, dip-coating, extrusion, thermoplastic extrusion, freeze casting, ink-jet printing, additive manufacturing, 3D printing, slurry coating, slip casting, and variants thereof.

In one embodiment of the present disclosure, the piezoelectric material for the proliferating layer has a piezoelectric constant, or dᵢⱼ, of between 25 pC/N and 120 pC/N, such as of 35-45 pC/N, such as of 40-55 pC/N, such as of 50-65 pC/N, such as of 60-75 pC/N, such as of 70-85 pC/N, such as of 80-95 pC/N, such as of 90-105 pC/N, such as of 100-115 pC/N, such as of at the most 120 pC/N.

In one embodiment of the present disclosure, the reagents for the virucidal layer comprise a piezoelectric material with a piezoelectric constant, or dᵢⱼ, of 150 pC/N or higher, such as of 165-175 pC/N, such as of 160-170 pC/N, such as of 175-185 pC/N, such as of 170-180 pC/N, such as of 185-195 pC/N, such as of 180-190 pC/N, such as of at the most 200 pC/N.

In one embodiment of the present disclosure, the reagents for the proliferating layer and/or for the virucidal layer also comprise a lubricant, a binder, a rheology controller, a pore former, a solvent, and/or a dispersant. A person of skill in the art is capable of a suitable reagents selection depending on the process used and based on common general knowledge in the field of ceramics.

In one embodiment of the present disclosure, steps a) and b) and thus fabrication of the proliferation layer, occur before steps c) and d), fabrication of the virucidal layer.

In one embodiment of the present disclosure, steps a) and b) and thus fabrication of the proliferation layer, occur at about the same time as steps c) and d), fabrication of the virucidal layer.

In one embodiment of the present disclosure, steps a) and b) and thus fabrication of the proliferation layer, occur after steps c) and d), fabrication of the virucidal layer.

In one embodiment of the present disclosure, step e) of heat-treating the proliferation layer may occur at the same time, before or after step f) of heat-treating the virucidal layer.

Thus, the first proliferation layer and the virucidal layer may be heat-treated together or separately.

In one embodiment of the present disclosure, steps e) and f) of heat-treating the first proliferation layer and the virucidal layer may occur after step g), thus after the layers have been set together.

In one embodiment of the present disclosure, step g) of sintering the proliferation layer may occur at the same time, before or after step h) of sintering the virucidal layer. Thus, the first proliferation layer and the virucidal layer may be sintered together or separately.

In one embodiment of the present disclosure, steps g) and h) of sintering the first proliferation layer and the virucidal layer may occur after step g), thus after the layers have been bound together and also after the layers have been heat-treated.

In one embodiment of the present disclosure, step i) comprises depositing the proliferation layer onto the virucidal layer, or depositing the virucidal layer onto the proliferation layer.

In one embodiment of the present disclosure, step i) comprises coating the proliferation layer with the virucidal layer, or coating the virucidal layer with the proliferation layer.

In one embodiment of the present disclosure, step i) comprises binding the proliferation layer to the virucidal layer by an adhesive layer.

In one embodiment of the present disclosure, the method comprises a step of poling the piezoelectric materials of the proliferation layer and/or virucidal layer.

In one embodiment of the present disclosure, the poling of the piezoelectric material of the proliferation layer comprises:
a) Infiltrating the proliferation layer with silicon oil;
b) Placing an electrode in the proliferation layer;
c) Heat-treating the proliferation layer above the curie temperature of the piezoelectric material used;
d) Applying a static electric field above the coercive field of the piezoelectric material used, and
e) Removing the electrode.

The poling of the piezoelectric material of the virucidal layer comprises:
a. Infiltrating the virucidal layer with silicon oil;
b. Placing an electrode between the proliferation layer and the virucidal layer and a second electrode in the virucidal layer;
c. Heat-treating the virucidal layer at a temperature curie temperature
d. Applying a static electric field above the coercive field of the piezoelectric material in used,
e. Removing the electrodes.

In one embodiment of the present disclosure, the poling is conducted on the whole fermentation enhancement device, for example by placing the whole fermentation enhancement device between two electrodes without being in direct contact with the two electrodes. For example, the fermentation enhancement device of the present disclosure may undergo Corona poling.

### DETAILED DESCRIPTION

The present disclosure will in the following be described in greater detail with reference to the accompanying drawings. The drawings are exemplary and are intended to illustrate some of the features of the presently disclosed fermentation enhancement device, bioreactor and related methods, and are not to be construed as limiting to the preset disclosure.

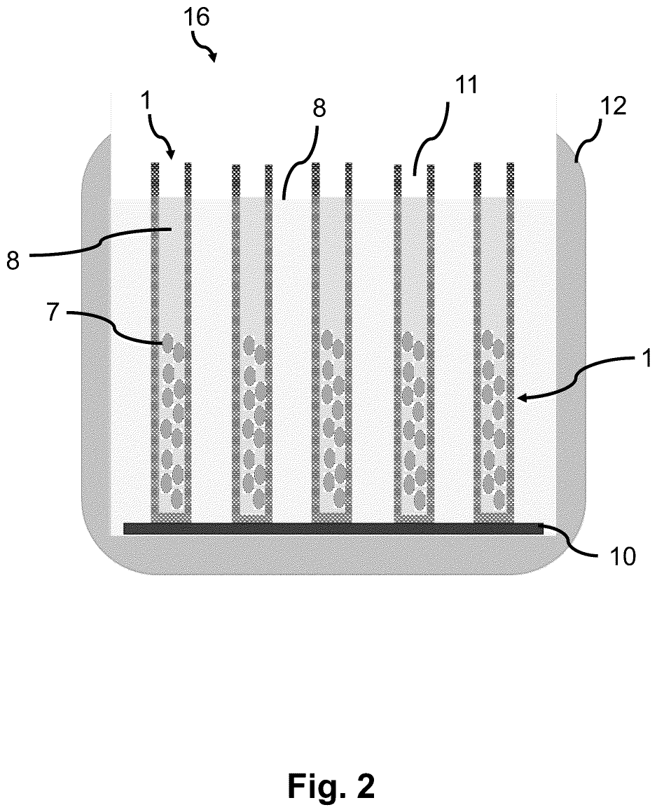

In the description below, the terms “top”, and “opening”, refer to the upright position of the fermentation enhancement device shown in FIGS. 1a-1c and 2.

FIGS. 1a-c shows a plurality of cross-sections of different views of a schematic illustration of an embodiment of the presently disclosed fermentation enhancement device (1). FIG. 1a shows a cross-section from a frontal view of the fermentation enhancement device (1). As can be seen, the fermentation enhancement device (1) comprises an electrical charge structure (14), that comprises a proliferation layer (3) located at an inner side of the electrical charge structure (14). The proliferation layer defines an inner volume (15) that is arranged to accommodate a fermentation process.

The proliferation layer (3) in this specific example is provided as a cylindrical container that is arranged to accommodate the fermenting microorganism (7) and culture media (8), wherein the culture media typically contains nutrients (9). The fermentation enhancement device (1) further comprises an opening (11). Typically, the electrical charge structure comprises the opening (11), it is arranged such that medium, nutrients and/or fermenting microorganism can cross the opening (11). For example, the opening (11) can be used to add or remove fermenting microorganisms, such as bacteria, e.g. a starter culture. In the specific example given in FIG. 1, the opening (11) is located at a first end of the electrical charge structure (14). Additional, or alternative, positions of an opening (11) may for example be the side of the electrical charge structure (14), typically such that medium and/or nutrients may flow through said opening (11).

As disclosed elsewhere herein the electrical charge structure (14) may be provided in many different shapes and arrangements. In the specific example shown in FIGS. 1a-1c, the electrical charge structure (14) is arranged as a container. However, in other examples (not shown), the second end of the electrical charge structure (14) may be covered by a substrate or an actuation source. Thus it is not necessary that the electrical charge structure (14) forms a container by itself.

Further, it should be noted, although the example shown illustrates the proliferation layer (3) and the virucidal layer (2) forming the electrical charge structure (14), wherein the proliferation layer (3) abuts the virucidal layer (2), the electrical charge structure (14) may comprise other layers, such as an intermediary electrode layer (not shown) or other materials. Thus, the proliferation layer may be provided as only a part of the inner surface of the electronic charge structure (14), and the virucidal layer (2) may be provided as only a part of the outer surface of the electronic charge structure (14).

As shown in FIGS. 1a-1c, in use, the fermentation enhancement device (1) comprises the fermenting microorganism, which could be yeast, algae, bacteria (7), e.g. LAB or another bacteria (7) having an extracellular electron transfer mechanism (EET), suspended in a culture media (8). Typically, a part of the electrical charge structure (14) is arranged to protrude above the surface of the culture medium (8), such that the fermenting microorganism (7) is contained in the culture medium (15) by the fermentation enhancement device (1). The fermentation enhancement device (1) may be arranged to be contained by a fermentation vessel, such as a bioreactor. The fermentation vessel may in such a case be arranged to contain the medium (8) that the fermentation enhancement device is, at least partly, submerged into.

As shown in FIG. 1b, actuation of an actuation source (not shown) results in the generation of electrical charges (4) and (5), respectively, on the proliferation layer (3) and the virucidal layer (2) of the electrical charge structure (14). Typically, the actuation source (10) and the electrical charge structure (14) is arranged such that an inner charge (4) is generated at the proliferation layer (3), while an outer charge (5) is generated at the virucidal layer (2). Typically, the inner charge (4) is smaller than the outer charge (5). The inner charge is typically configured to increase bacterial growth, while the outer charge is configured to kill and/or to inactivate bacteriophages, other viruses, and/or microorganisms for example through formation of ROS and associated redox reactions. The specific example shown, comprises an electrical charge structure (14) that allows for passage of nutrients (9) while preventing the passage of bacteriophages (6). This may for example be achieved by having a porous proliferation layer and a porous virucidal layer, wherein the smallest pore size is selected to exclude passage of bacteriophages (6), but permits passage of nutrients (9).

FIG. 1c shows a cross-section of the same device as shown in FIG. 1a-1b, along its axial length. The electrical charge structure (14) can be seen to have a circular cross section, and comprises the proliferation layer (3) and the virucidal layer (2). The outer charges act to kill and/or inactivate bacteriophages (6), other viruses, and/or electrogenic microorganisms while the inner charges (4) act to stimulate growth of the fermenting microorganism (7).

FIG. 2 is a schematic illustration of one embodiment of the presently disclosed fermentation system (16) comprising a plurality of fermentation enhancement devices (1). The fermentation enhancement devices (1) of this specific example are arranged in an array inside a fermentation vessel (12). In this example, the fermentation vessel is an open vessel, but the vessel may be arranged to form a sealed environment for better control of for example temperature, gasses and pH.

The fermentation system comprises an actuation source (10), which may be in the form of a piezoelectric transducer arranged to provide vibration, e.g. ultrasonic vibrations, to the electrical charge structure. The actuation source may be abutting the electrical charge structure or coupled to the electrical charge structure through other structures, such as a substrate (not shown)

In this example, a single actuation source (10) is arranged to actuate multiple fermentation enhancement devices (1). The actuation source (10) abuts each of said multiple fermentation enhancement devices (1). The fermentation vessel (12) contains the fermentation enhancement devices (1) such that, during use, the fermentation enhancement devices (1) may be submerged, at least partly, by the medium. Thus, the opening of each fermentation enhancement device, may be above the medium. As disclosed elsewhere herein, in other examples, the opening may however be arranged to be below the surface of the medium. In specific examples, the fermentation system may comprise fermentation enhancement devices configured as the example given in FIGS. 1a-1c.

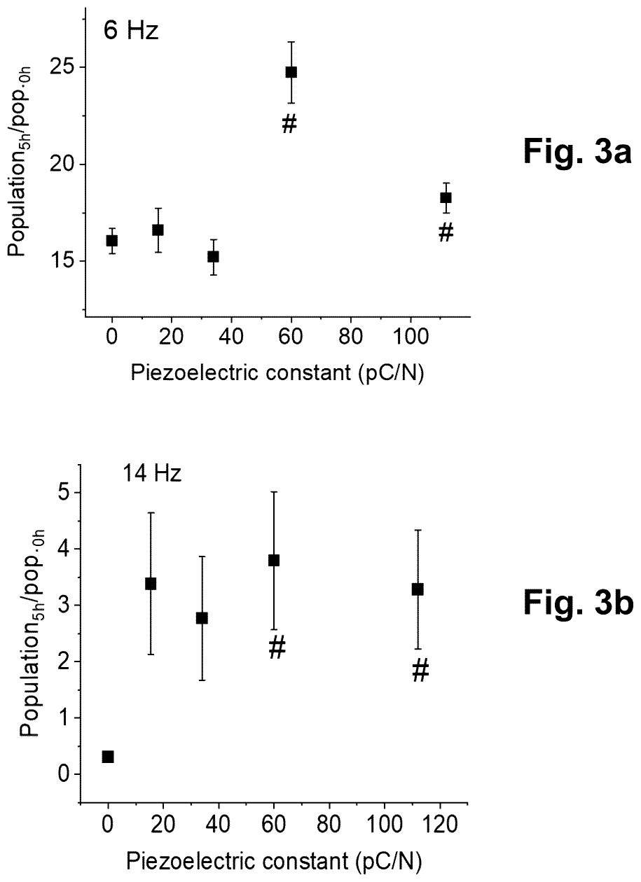

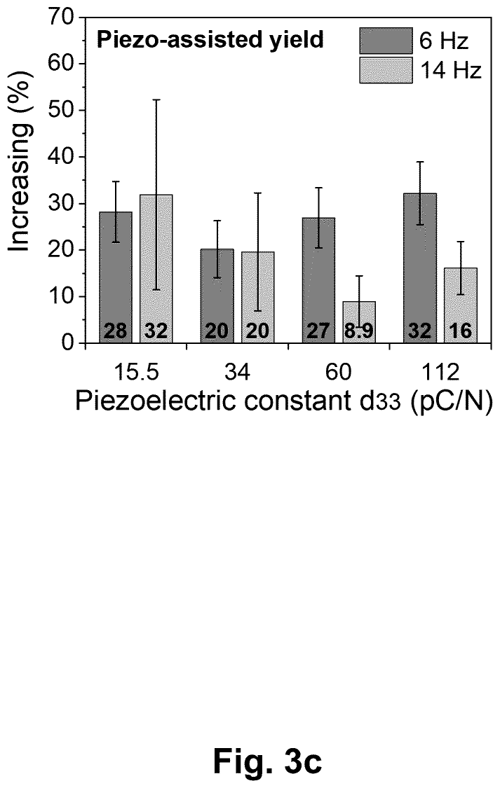

FIGS. 3a-3c show experimental results from using a fermentation system according to one embodiment of the present disclosure, for fermentation of lactic acid bacteria (LAB). FIGS. 3a and b shows the proliferation of L. lactis after 5 hours, depending on the piezoelectric constant of the material.

The values reported are the ratio of CFU count after 5 hours and CFU at the beginning of the (CFU/CFU0). At both 6 and 14 Hz, an increase in the population is observed for the fermentation enhancement device with d₃₃=60 and 115 pC/N. The values are statistically significant (#) with confidence >95%, determined with a two-tail ANOVA test.

FIG. 3a shows in particular for 6 Hz, the population in 5 hours increased from a factor of 15 on the non-piezoelectric device to a factor of 25 on the piezoelectric fermentation enhancement device (66% increase). FIG. 3b shows at 14 Hz, the growth was inhibited by vibrations, but in piezoelectric fermentation enhancement devices, the growth was 6× higher with respect to non-piezo devices. FIG. 3c shows lactate yield increase in piezoelectric fermentation enhancement devices with respect to the non-piezo ones at 6 Hz and 16 Hz.

### EXAMPLES

### Example 1. Manufacture of a Dual Functional Fermentation Enhancement Device

The proliferation layer may be fabricated by thermoplastic extrusion. The feedstock for the extrusion may be prepared by mixing the piezoelectric powder (e.g., BaTiO₃), lubricant (e.g., stearic acid and wax), binder (e.g., Ethylene vinyl acetate), rheology controllers (e.g., wax) and pore formers (e.g., polymethyl methacrylate). The feedstock is mixed and then extruded at 110 °C. The tubes obtained may then be treated with a thermal process: 2 hours at 120 °C, 250 °C, 450 °C and 650 °C to remove organics and pore formers and then 2 hours at 1350 °C for the powder sintering (rate of 30 °C/h). This is only an exemplary protocol and not limiting for the present disclosure.

Alternative processes that can be used to manufacture the proliferative layer are e.g. tape casting and screen printing. In order to induce the piezoelectric properties, the sintered BaTiO₃ materials may need to be poled. For the poling process, the BaTiO₃ tube may be infiltrated with silicon oil and electrodes may be placed on the inner and outer sides. The fermentation enhancement device may then be heated at a temperature higher than the BaTiO₃ curie temperature, for example at around 140 °C, and a static electric field of for example 1.5-2 kV/cm may be applied to the sample for around half an hour. During the application of voltage, the temperature may be gradually lowered (−60 °C/h). After that, the piezoelectric effect may be measured with a piezotester. The inner electrode is then removed with acetone.

For the integration of the virucidal piezoelectric layer, the proliferation layer may be dip-coated with a slurry consisting of piezoelectric power (e.g., BaTiO₃) or ceramic polymer precursors, solvent (e.g., water and/or ethanol), binder (e.g., polyvinyl alcohol) and dispersant (e.g., polyvinylpyrrolidone). After coating with the virucidal layer, the bi-layer electrical charge structure may be subjected to heat treatment for example at 600 °C or less for approximately 4 hours, to remove the organic additives. Subsequently, the electrical charge structure may be sintered at temperatures between e.g. 800-1400 °C for approximately 2 hours in presence of air.

Alternative processes that can be used to obtain the virucidal layer are for example sol-gel coating and spray coating. The poling may be repeated for the virucidal layer with similar conditions. This step, however, requires contacting the electrode between the proliferation layer and the virucidal layer and the one on the virucidal layer. Also, the electric field applied may be in the range of e.g. 5-6 kV/cm to obtain a higher piezoelectric coefficient.

### Example 2. Performance of a Dual Functional Fermentation Enhancement Device

We present here the bacterial proliferation of L. lactis in a BaTiO₃ piezoelectric fermentation enhancement device, a fermentation enhancement device of the present disclosure. The evaluation includes growth rate, and lactose production yield. The investigation was carried out on dense piezoelectric surfaces, to demonstrate the proliferation of the lactic acid bacteria. However, porous piezoelectric surfaces are also suitable. The BaTiO₃ samples were poled as described previously.

The strain Lactococcus lactis cremoris MG1363 was cultivated on M17 broth with 1% glucose. The culture was first incubated overnight at 30 °C and agitated at 200 rpm. Then, the culture optical density (OD600) was set to 0.05 and placed a suspension of 350 μL on the positively polarized surface of the piezoelectric device. Fermentation enhancement devices with different piezoelectric coefficients were used. In addition, a non-piezoelectric BaTiO₃ reactor was used as a reference. The fermentation enhancement devices were closed in a plastic box to avoid disturbances from the surroundings and placed on a vibration source. The vibration was controlled with a voltage generator and set to an amplitude of 1 mm (triangle wave) at 6 and 14 Hz, with 1 N force. Another set of plastic plates and BTO samples were kept in static condition as a reference. The process was carried out at room temperature and for 5 hours.

The viability of bacterial cells before and after the experiment was assessed by colony forming unit (CFU) count. To quantify lactic acid production yield High-pressure liquid chromatography (HPLC) measurements were used. HPLC was performed on an Ultimate 3000 high-pressure liquid chromatography system (Dionex, Sunnyvale, CA) with an Aminex HPX-87H column (Bio-Rad, Hercules, CA) and a Shodex RI-101 detector (Showa Denko K.K., Tokyo, Japan). The column oven temperature was set at 60 °C, the mobile phase was at 5 mM H₂SO₄, and the flow rate was 0.5 ml/min.

Results: FIGS. 3a and 3b shows the proliferation of L. lactis after 5 hours, depending on the piezoelectric constant of the fermentation enhancement device. The values reported are the ratio of CFU count after 5 hours and CFU at the beginning of the experiment (CFU/CFU0). At both 6 and 14 Hz, an increase of the population is observed for the reactors with d₃₃=60 and 115 pC/N. The values are statistically significant (#) with a confidence >95%, determined with a two-tails ANOVA test. In particular, for 6 Hz, the population in 5 hours increased from a factor of 15 on the non-piezoelectric reactor (consistent with literature) to a factor of 25 on the piezoelectric fermentation enhancement device (66% increase). At 14 Hz, the bacteria was subjected to inhibited growth conditions for all devices (both non-piezo and piezoelectric). The piezoelectric action avoided the growth reduction of bacteria and an increased proliferation is observed for the bacteria placed in piezoelectric fermentation enhancement devices with d₃₃=60 and 115 pC/N.

FIG. 3c shows the HPLC yield measurements for the same samples and the same frequencies of vibration. The lactate yield is defined as lactate production in mM divided by the glucose consumption in mM:

$$Yield = \frac{m {M}_{lactate}}{{mM}_{glucose consumed}}$$

The values reported are the ratio of the lactate yield of bacteria placed on piezoelectric fermentation enhancement devices and yield in non-piezo devices:

40

$$Piezoaction = \frac{{Yield}_{piezo}}{{Yield}_{{nonpiezo}_{}}}$$

At both frequencies, the piezoelectric reactors increased the yield by up to 50%. Interestingly, at 14 Hz a higher lactate yield increase was observed, and the opposite at 6 Hz.

Conclusion: The impact of the piezoelectric charges on L. lactis proliferation and lactate production is clearly demonstrated on the variable piezoelectric surfaces. The data did not provide a clear dependency on the frequency of the piezoelectric constant; however, the piezoelectric fermentation enhancement devices induced on average higher growth of the bacteria culture. For optimal growth conditions, 66% higher proliferation was achieved. The lactate production yield is also increased with the use of piezoelectric fermentation enhancement devices, with an increase of up to 15% at 6 Hz and 50% at 14 Hz. Without being bound to theory, it is possible that EET facilitates the electron transfer process from piezoelectric surfaces to the cell membranes and so contributes to the proliferation process of microorganism having EET, such as L. lactis.

### REFERENCES

S. Tejedor-Sanz et al., “Extracellular electron transfer increases fermentation in lactic acid bacteria via a hybrid metabolism,” Elife, vol. 11: e70684 (2022).
S. Freguia, M. Masuda, S. Tsujimura, and K. Kano, “Lactococcus lactis catalyses electricity generation at microbial fuel cell anodes via excretion of a soluble quinone,” Bioelectrochemistry, vol. 76, no. 1-2, pp. 14-18 (2009).
Yu, X., Wang, S., Zhang, X., Qi, A., Qiao, X., Liu, Z., Wang, Z. L., “Heteroelectrical charge structured nanorod array with piezophototronic and plasmonic effect for photodynamic bacteria killing and wound healing”. Nano Energy, 46, 29-38 (2018).
Tan, G., Wang, S., Zhu, Y., Zhou, L., Yu, P., Wang, X., Ning, C. “Surface-Selective Preferential Production of Reactive Oxygen Species on Piezoelectric Ceramics for Bacterial Killing”. ACS Applied Materials and Interfaces, 8(37), 24306-24309 (2016).
Carvalho, E. O., Fernandes, M. M., Padrao, J., Nicolau, A., Marqués-Marchán, J., Asenjo, A., Lanceros-Mendez, S. “Tailoring Bacteria Response by Piezoelectric Stimulation”. Applied Materials & Interfaces, 11(30), 285-287 (2019).
https://www.statista.com/statistics/754737/dairy-processing-equipment-market-value-in-the-us-by-type/Dupré-Crochet,
S., Erard, M. and Nüβe, O. ROS production in phagocytes: why, when, and where?. Journal of Leukocyte Biology, 94: 657-670 (2013).
Xu, Q., Gao, X., Zhao, S., Liu, Y.-N., Zhang, D., Zhou, K., Khanbareh, H., Chen, W., Zhang, Y., Bowen, C., Construction of Bio-Piezoelectric Platforms: From Structures and Synthesis to Applications. Adv. Mater., 33, 2008452 (2021).
Egusa, S., Wang, Z., Chocat, N. et al. “Multimaterial piezoelectric fibres”. Nature Mater 9, 643-648 (2010).
Brown, C., Kell, R., Taylor, R., and Thomas, L:, “Piezoelectric Materials, A Review of Progress,” in IRE Transactions on Component Parts, vol. 9, no. 4, pp. 193-211 (1962).
Tamang, JP., Cotter, P., Endo, A., et al. “Fermented foods in a global age: East meets West”. Compr Rev Food Sci Food Saf.; 19:184-217 (2020).
Gänzle, M. “The periodic table of fermented foods: limitations and opportunities”. Appl Microbiol Biotechnol 106, 2815-2826 (2022).
Thapa et al. 2022. Overview of electroactive microorganisms and electron transfer mechanisms in microbial electrochemistry. Bioresource Technology 347, 126579.
Garbini et al. 2023. Electroactive Bacteria in Natural Ecosystems and Their Applications in Microbial Fuel Cells for Bioremediation: A Review. Microorganisms 2023, 11(5), 1255.
Singh and Kumar, 2022. Chapter 27—Bioelectrochemical system for environmental remediation of toxicants, Microbial Biodegradation and Bioremediation (Second Edition).

1. A fermentation enhancement device for use in a bioreactor, the fermentation enhancement device comprising:
an electrical charge structure defining an inner volume arranged for accommodating a fermentation process, the electrical charge structure comprising:
a piezoelectric proliferation layer exposed to the inner volume; and
a piezoelectric virucidal layer arranged at an outer surface of the electrical charge structure; and
an opening configured to allow therethrough a flow of nutrients and/or fermenting microorganism;
wherein the fermentation enhancement device is arranged such that actuation of the electrical charge structure, by an actuation source, generates an electrical charge at the virucidal layer that kills and/or inactivates viruses and/or microorganisms, and an electrical charge at the proliferation layer that stimulates growth of fermenting microorganisms.

2. The fermentation enhancement device according to claim 1, wherein the electrical charge structure is an elongated structure, such as a cylindrical structure.

3. The fermentation enhancement device according to any of the preceding claims, wherein the electrical charge structure is a container.

4. The fermentation enhancement device according to any of the preceding claims, wherein the electrical charge structure has an inner surface that is, partly or fully, covered by the proliferation layer.

5. The fermentation enhancement device according to any of the preceding claims, wherein the electrical charge structure has an outer surface that is, partly or fully, covered by the virucidal layer.

6. The fermentation enhancement device according to any of the preceding claims, wherein the virucidal layer is arranged at an outer surface of the electrical charge structure and the proliferation layer is arranged at an inner surface of the electrical charge structure.

7. The fermentation enhancement device according to any of the preceding claims, wherein an inner surface of the electrical charge structure is made of the proliferation layer, and wherein the outer surface of the electrical charge structure is made of the virucidal layer.

8. The fermentation enhancement device according to any of the preceding claims, wherein the electrical charge structure is provided on a support layer.

9. The fermentation enhancement device according to any of the preceding claims, wherein a cross section of the electrical charge structure, along its axial length, such as from a first end to a second end, is rounded, such as circular, or a polygonal, such as rectangular or squared.

10. The fermentation enhancement device according to any of the preceding claims, wherein the virucidal layer abuts the proliferation layer.

11. The fermentation enhancement device according to any of the preceding claims, wherein the virucidal layer and the proliferation layer are separated by one or more further layers, such as an electrode layer of a conducting material.

12. The fermentation enhancement device according to any of the preceding claims, wherein a first end of the electrical charge structure is covered by the proliferation layer and/or the virucidal layer.

13. The fermentation enhancement device according to any of the preceding claims, wherein the fermentation enhancement device comprises multiple openings.

14. The fermentation enhancement device according to any of the preceding claims, wherein a first end of the electrical charge structure comprises an opening or is covered by a substrate.

15. The fermentation enhancement device according to any of the preceding claims, wherein a second end of the electrical charge structure comprises the opening.

16. The fermentation enhancement device according to any of the preceding claims, wherein the proliferation layer is thicker than the virucidal layer, or the virucidal layer is thicker than the proliferation layer, or wherein the proliferation layer and the virucidal layer have about the same thickness.

17. The fermentation enhancement device according to any of the preceding claims, wherein the virucidal layer has a thickness of 1 nm or more, such as a thickness of 5 nm or more.

18. The fermentation enhancement device according to any of the preceding claims, wherein the electrical charge structure has an inner diameter of at least 0.5 mm and at the most 20 mm.

19. The fermentation enhancement device according to any of the preceding claims, wherein the electrical charge structure has a thickness of at least 5 nm and at the most 20 mm.

20. The fermentation enhancement device according to any of the preceding claims, wherein the proliferation layer comprises a piezoelectric material.

21. The fermentation enhancement device according to any of the preceding claims, wherein the virucidal layer comprises a piezoelectric material.

22. The fermentation enhancement device according to any of the preceding claims, wherein the proliferation layer and the virucidal layer comprise different piezoelectric materials.

23. The fermentation enhancement device according to any of the preceding claims, wherein the proliferation layer and the virucidal layer comprise a same piezoelectric material.

24. The fermentation enhancement device according to any of the preceding claims, wherein the proliferation layer and the virucidal layer have different chemical and/or physical properties, such as different porosity, and/or different piezoelectric coefficient (dᵢⱼ).

25. The fermentation enhancement device according to any of the preceding claims, wherein the proliferation layer comprises a piezoelectric material that has a piezoelectric constant between 25 pC/N and 120 pC/N.

26. The fermentation enhancement device according to any of the preceding claims, wherein the virucidal layer piezoelectric material has a piezoelectric constant of 150 pC/N or higher.

27. The fermentation enhancement device according to any of the preceding claims, wherein the proliferation layer and the virucidal layer are non-porous.

28. The fermentation enhancement device according to any of the preceding claims, wherein the proliferation layer is porous and virucidal layer is non-porous.

29. The fermentation enhancement device according to any of the preceding claims, wherein the proliferation layer is non-porous and virucidal layer is porous.

30. The fermentation enhancement device according to any of the preceding claims, wherein both the proliferation layer and virucidal layer are porous.

31. The fermentation enhancement device according to any of the preceding claims, wherein the proliferation layer is porous, and wherein the pores within the proliferation layer have a size of 100 μm or less, such as a size of between 10-20 μm.

32. The fermentation enhancement device according to any of the preceding claims, wherein the proliferation layer is porous, and wherein the proliferation layer has a total porosity of at least 5% by volume and at the most 70% by volume, preferably at least 30% by volume, such as of about 35% by volume.

33. The fermentation enhancement device according to any of the preceding claims, wherein the virucidal layer is porous, and wherein the pores within the virucidal layer have a size of between 5 and 100 nm, such as a size of 15 nm or less.

34. The fermentation enhancement device according to any of the preceding claims, wherein the virucidal layer has a total porosity of at least 5% by volume and at the most 70% by volume, preferably at least 20% by volume, such as of about 30% by volume.

35. The fermentation enhancement device according to any of the preceding claims, wherein the device is arranged such that the charge generated, during use, at the proliferation layer is different from the charge at the virucidal layer.

36. The fermentation enhancement device according to any of the preceding claims, wherein the fermentation enhancement device is arranged such that actuation of the electrical charge structure generates a charge at the proliferation layer, and wherein said charge stimulates growth of fermenting microorganisms.

37. The fermentation enhancement device according to any of the preceding claims, wherein the fermentation enhancement device is arranged such that actuation of the electrical charge structure generates a charge at the virucidal layer that generates reactive oxygen species (ROS).

38. The fermentation enhancement device according to claim 37, wherein the fermentation enhancement device is arranged such that the charge at the virucidal layer kills and/or inactivates bacteriophages through formation of ROS and associated redox reactions.

39. The fermentation enhancement device according to any of the preceding claims, wherein the fermentation enhancement device is arranged such that the charge at the virucidal layer kills and/or inactivates prokaryotic microorganisms through formation of ROS and associated redox reactions.

40. The fermentation enhancement device according to any of the preceding claims, wherein the fermentation enhancement device is arranged such that the charge at the virucidal layer kills and/or inactivates eukaryotic microorganisms through formation of ROS and associated redox reactions.

41. The fermentation enhancement device according to any of the preceding claims, wherein the prokaryotic microorganisms are bacteria and/or archaea.

42. The fermentation enhancement device according to any of the preceding claims, wherein the eukaryotic microorganisms are fungi and/or algae.

43. The fermentation enhancement device according to any of the preceding claims, wherein the fermentation enhancement device is suitable for culturing bacteria, such as gram-negative bacteria, or gram-positive bacteria, such as bacteria having extracellular electron transfer mechanism (EET).

44. The fermentation enhancement device according to any of the preceding claims, wherein the fermenting microorganisms are bacteria, such as gram-negative bacteria, or gram-positive bacteria, such as bacteria having extracellular electron transfer mechanism (EET).

45. The fermentation enhancement device according to any of the preceding claims, wherein the fermenting microorganisms are eukaryotic microorganisms, such as fungi or algae, such as eukaryotic microorganisms having extracellular electron transfer mechanism (EET).

46. The fermentation enhancement device according to any of the preceding claims, wherein said fermentation enhancement device is arranged for being contained by a bioreactor.

47. A fermentation system comprising:
one or more fermentation enhancement device(s) according to any of the preceding claims;
one or more actuation source(s) arranged to actuate the electrical charge structure(s) of the fermentation enhancement device(s); and
a fermentation vessel accommodating the fermentation enhancement device(s).

48. The fermentation system according to claim 47, wherein the fermentation system comprises a plurality of fermentation enhancement devices.

49. The fermentation system according to any of claims 47-48, wherein the fermentation system comprises a plurality of actuation sources, such as wherein each fermentation enhancement device is arranged to be actuated by a different actuation source.

50. The fermentation system according to any of claims 47-49, wherein a plurality of fermentation enhancement devices are arranged to be actuated by a single actuation source.

51. The fermentation system according to any of claims 47-50, wherein the actuation source is a transducer, such as a piezoelectric transducer, an agitator and/or a shaker, preferably wherein the actuation source is arranged to provide vibrations to the electrical charge structure.

52. The fermentation system according to any of claims 47-51, wherein the actuation source is in physical contact with the electrical charge structure, such as wherein the actuation source abuts the electrical charge structure.

53. The fermentation system according to any of claims 47-52, wherein the actuation source(s) are arranged to provide a vibration force of between 0.1-20 N, such as between 1-4 N, preferably at low frequency, such as 5-20 Hz.

54. The fermentation system according to any of claims 47-53, wherein two or more fermentation enhancement devices are positioned on a same horizontal plane inside the fermentation vessel.

55. The fermentation system according to any of claims 47-54, wherein two or more fermentation enhancement devices are positioned on different horizontal planes inside the fermentation vessel.

56. The fermentation system according to any one of claims 47-55, wherein the fermentation vessel forms a sealed environment around the fermentation enhancement device(s).

57. The fermentation system according to any of claims 47-56, wherein the fermentation system is a bioreactor.

58. A method for production of a fermented product, said method comprising:
adding, to a fermentation system according to any one of claims 47-57, a starter culture of a fermenting microorganism and a suitable culture medium;
operating the fermentation system, including actuation of the actuation source such that electrical charges are generated at the electrical charge structure comprising an outer charge generated at the virucidal layer that kills and/or inactivates viruses and/or microorganisms, and an inner electrical charge generated at the proliferation layer that stimulates growth of the fermenting microorganism.

59. The method according to claim 58, wherein the electrical charge structure(s) of the one or more fermentation enhancement devices are at least partially submerged in the culture medium, such as wherein the electrical charge structures of the one or more fermentation enhancement devices are submerged in the culture medium.

60. The method according to any one of claims 58 to 59, wherein the step of operating comprises actuating the electrical charge layer by the actuation source, and providing a suitable atmosphere for culturing of the fermenting microorganism.

61. The method according to any one of claims 58 to 60, wherein the fermenting microorganism is a prokaryotic microorganism or an eukaryotic microorganism.

62. The method according to any one of claims 58 to 61, wherein the prokaryotic microorganism is a bacteria and/or an archaea.

63. The method according to any one of claims 58 to 62, wherein the prokaryotic microorganism is a bacteria, such as wherein the bacteria is a gram-positive or a gram-negative bacteria, such as wherein the bacteria is a bacteria with EET.

64. The method according to any one of claims 58 to 63, wherein the gram-positive bacteria is selected from the group consisting of: lactic acid bacteria, such as Lactococcus, Enterococcus, Streptococcus, Pediococcus, Leuconostoc, Oenococcus, Weissella, and group I and group III lactobacilli.

65. The method according to any one of claims 58 to 61, wherein the eukaryotic microorganism is a fungi and/or an algae, such as wherein the eukaryotic microorganism is a fungi with EET and/or an algae with EET.

66. The method according to any one of claims 58 to 65, wherein the culture medium comprises milk, such as cow milk, goat milk, sheep milk.

67. The method according to any one of claims 58 to 66, wherein the fermented product is a fermented diary product.

68. The method according to any one of claims 58 to 65, wherein the fermented product is fermented fish.

69. The method according to any one of claims 58 to 65, wherein the fermented product is fermented meat.

70. The method according to any one of claims 58 to 65, wherein the fermented product is fermented vegetables.

71. The method according to any one of claims 58 to 65, wherein the fermented product is fermented legumes.

72. The method according to any one of claims 58 to 65, wherein the fermented product is fermented staple foods, such as wherein the fermented product is fermented as bread, or fermented cereal porridges.

73. The method according to any one of claims 58 to 65, wherein the fermented product is fermented coffee.

74. The method according to any one of claims 58 to 65, wherein the fermented product is fermented cocoa.

75. The method according to any one of claims 58 to 65, wherein the fermented product is fermented sauce, such as fermented vinegar or fermented soy sauce.

76. The method according to any one of claims 58 to 65, wherein the fermented product is a fermented beverage, such as a fermented alcoholic beverage.

77. A method for fabricating a fermentation enhancement device according to any one of claims 1-47, the method comprising:
a) providing proliferation layer reagents comprising a piezoelectric material;
b) mixing the proliferation layer reagents and fabricating the proliferation layer;
c) providing virucidal layer reagents comprising a piezoelectric material;
d) mixing the virucidal layer reagents and fabricating the virucidal layer;
e) heat-treating the proliferation layer;
f) heat-treating the virucidal layer;
g) sintering the proliferation layer;
h) sintering the virucidal layer;
i) binding the proliferation layer with the virucidal layer to form an electrical charge structure that is arranged such that actuation of said structure, by an actuation source, generates an electrical charge at the virucidal layer that kills and/or inactivates viruses and/or microorganisms, and an electrical charge at the proliferation layer that stimulates growth of a fermenting microorganism;
thereby obtaining a fermentation enhancement device according to any one of claims 1-47.

78. The method according to claim 77, wherein steps a) and b) occur before, at the same time or after steps c) and d).

79. The method according to any of the claims 77 to 78, wherein step e) of heat-treating the proliferation layer may occur at the same time, before or after step f) of heat-treating the virucidal layer.

80. The method according to any of the claims 77 to 79, wherein step g) of sintering the proliferation layer may occur at the same time, before or after step h) of sintering the virucidal layer.

81. The method according to any of the claims 77 to 80, wherein steps g) and h) of sintering the proliferation layer and the virucidal layer occur after step g), thus after the layers have been bound together.

82. The method according to any of the claims 77 to 81, wherein step i) comprises depositing the proliferation layer onto the virucidal layer, or depositing the virucidal layer onto the proliferation layer.

83. The method according to any of the claims 77 to 82, wherein step i) comprises coating the proliferation layer with the virucidal layer, or coating the virucidal layer with the proliferation layer.

84. The method according to any of the claims 77 to 83, wherein the proliferation layer and/or the virucidal layer is fabricated by a particulate-based process, a precursor-based process or a miscellaneous process.

85. The method according to any of the claims 77 to 84, wherein the piezoelectric material for the proliferating layer has a piezoelectric constant of between 25 pC/N and 120 pC/N.

86. The method according to any of the claims 77 to 85, wherein the reagents for the virucidal layer comprise a piezoelectric material.

87. The method according to any of the claims 77 to 86, wherein the piezoelectric material for the virucidal layer has a piezoelectric constant of 150 pC/N or higher.

88. The method according to any of the claims 77 to 87, wherein the reagents for the proliferating layer and/or for the virucidal layer comprise a lubricant, a binder, a rheology controller, a pore former, a solvent, and/or a dispersant.

89. The method according to any of the claims 77 to 88, wherein the method comprises a step of poling the piezoelectric materials of the proliferation layer and/or virucidal layer.

90. The method according to any of the claims 77 to 89, wherein poling the piezoelectric material of the proliferation layer comprises:
Infiltrating the proliferation layer with silicon oil;
Placing an electrode in the proliferation layer;
Heat-treating the proliferation layer at above the curie temperature of the piezoelectric material used;
Applying a static electric field above the coercive field of the piezoelectric material used; and
Removing the electrode.

91. The method according to any of the claims 77 to 90, wherein poling the piezoelectric material of the virucidal layer comprises:
Infiltrating the virucidal layer with silicon oil;
Placing an electrode between the proliferation layer and the virucidal layer and a second electrode in the virucidal layer;
Heat-treating the virucidal layer at a temperature curie temperature
Applying a static electric field above the coercive field of the piezoelectric material in used;
Removing the electrodes.

92. The method according to any of the claims 77 to 91, wherein poling is conducted on the fermentation enhancement device as a whole, such as on the proliferation layer and on the virucidal layer simultaneously, such as by Corona poling.
